(19) United States
(12) Patent Application Publication
(10) Pub. No.: US 2017/0369927 A1
(43) Pub. Date: Dec. 28, 2017

(54) METHOD AND DEVICE FOR QUANTIFICATION OF TARGET MOLECULES

(71) Applicant: Chimera Biotec GmbH, Dortmund (DE)

(72) Inventors: Michael Adler, Langen-Debstedt (DE); Hendrik Schroeder, Olfen (DE); Mark Spengler, Bremen (DE)

(21) Appl. No.: 15/633,804

(22) Filed: Jun. 27, 2017

(30) Foreign Application Priority Data
Jun. 27, 2016 (EP) 16 176 473.3

(51) Int. Cl. C12Q 1/68; G01N 33/68

(52) U.S. Cl. CPC C12Q 1/6804; G01N 33/6869; G01N 2458/10; G01N 2333/5412

(57) ABSTRACT

A method for quantifying a plurality of target molecules in a sample may include releasing a target molecule from a non-covalent bond of a conjugate by using a fusion molecule. A kit may include a detection conjugate, a release reagent, nucleic acid amplification agents, and an amplification detection probe. A device may be designed to perform the methods.

### CROSS REFERENCE TO RELATED APPLICATIONS

This application claims priority to European Patent Application Serial No. 16 176 473.3 filed on Jun. 27, 2016, which is incorporated by reference herein in its entirety.

### FIELD OF THE INVENTION

The present invention lies in the field of biochemistry and relates to a method for quantifying a plurality of target molecules in a sample. The present invention also relates to the a method of releasing a target molecule from a conjugate by using a fusion molecule, a kit comprising a detection conjugate, a release reagent and nucleic acid amplification agents and to a device to perform the method of the invention.

### BACKGROUND

Molecular methods and diagnostic tests need to have a very high degree of sensitivity and specificity to provide a valuable tool in determining the presence of low concentrations of target molecules and molecular markers in the early stages of a disease. For an improved analysis of the data, it is advantageous if the method or test allows the (precise) quantification of the molecule of interest. There are a number of molecules present within serum, for example, interleukins and parathyroid hormone related protein, which are potential markers of cancer and other pathological conditions. Currently these molecules are only detectable during the late stages of the disease when they are overexpressed by malignancies. Under normal conditions the proteins are present at subpicomolar concentrations, i.e. 0.1 pM and less.

Furthermore, the early detection of pathogenic organisms in an infection can be critical to whether or not an infected subject survives. This is particularly the case in diseases such as bacterial meningitis and septicemia caused, for example by Staphyloccocus aureaus. The earlier and the more precise these molecules can be determined during the disease process the better the prognosis. However, early detection means that the molecules are at low concentrations and the signaling/quantitation systems of current immunoassays, using enzymes and chemiluminescence, does not provide sufficient sensitivity to measure at these low levels or only provide imprecise.

One possibility to detect molecules of interest with high sensitivity is the application of immuno-PCR (IPCR). Herein, the target molecule is coupled to a conjugate of a binding molecule, specific for the target, and marker DNA (“detection conjugate”) (for example, multimeric detection conjugates comprising several binding molecules and several DNA markers).

The binding molecule (for example, a specific antibody, but alternatively a receptor or an antigen, e.g. if the detected target molecule itself is an antibody) recognizes and binds the target molecule. The marker DNA is subsequently amplified (for example, in a PCR reaction, but alternatively also in an isothermal amplification reaction; detected by a DNA amplification signal-generating probe, for example a TaqMan probe, which is degraded during the extension of a complementary DNA strand by the exonuclease activity of the Taq-polymerase and generates a fluorescent signal) and thus provides for signal generation and signal amplification.

Critical for the quality of the IPCR is the separation of specifically bound detection conjugate and non-specific, unbound detection conjugate, as the presence of unspecifically bound detection conjugate generates a background signal. To reduce the background signal, the immuno-PCR is may be carried out in the sandwich assay format using a solid substrate and capture molecules.

In such a sandwich assay, a capture binding molecule, which binds the target molecule and—at the same time (“combined”) or in two separate steps (“sequential”)—the detection conjugate, is immobilized on a surface. Before addition of the signal generating reagents (e.g. PCR agents and probe), a stringent wash step is carried out which removes non-specifically bound detection conjugate. Additional suitable washing and blocking steps to minimize non-specific interactions can be applied subsequently to the respective incubation steps. In addition, the target molecule can be diluted with a suitable buffer to minimize matrix effects (e.g. of biological sample material).

Although the IPCR method provides a sensitive method to detect a molecule of interest, it is still difficult, time consuming and expensive to quantify the amount of the molecules of interest present in the original sample due to the above-mentioned issues. Hence, there is need in the art for an improved method that allows the sensitive and precise quantification of a molecule of interest.

### SUMMARY

The present invention meets the above need by providing a method for quantifying a plurality of target molecules, as described herein. Surprisingly, the present inventors found that the advantages of immuno-PCR (IPCR), such as high sensitivity, can be connected with the advantages of digital-PCR, such as precise quantification, by combining both of these methods. To achieve this, either the complete IPCR detection conjugate or the nucleic acid marker needs to be isolated, i.e. typically eluted from a substrate, for further processing. Surprisingly, the present inventors have found that the elution of a plurality of nucleic acid markers can be efficiently achieved by adding a release agent which interferes with a non-covalent bond between the nucleic acid marker and the other components of the detection conjugate and thus results in the release of the nucleic acid marker from the remaining components of the detection conjugate. Thereby, a plurality of nucleic acid markers is eluted from the surface-bound detection conjugate/target molecule complex and can be used in a plurality of parallel amplification reactions to quantify of the amount of target molecules present in the original sample.

In a first aspect, the present invention is thus directed to a method for quantifying a plurality of target molecules in a sample, comprising a) providing (i) the plurality of target molecules, wherein the target molecules are immobilized on a solid substrate, and (ii) a plurality of detection conjugates each comprising a binding molecule binding specifically to the target molecule and a nucleic acid marker, wherein the binding molecule and the nucleic acid marker are linked by non-covalent binding, b) contacting the plurality of target molecules and the plurality of detection conjugates, such that the detection conjugates bind to the target molecules, c) adding (i) a release agent that releases the target molecules from the binding molecules and the nucleic acid markers of the plurality of detection conjugates by competitive binding to at least one member of the non-covalent bond, and (ii) nucleic acid amplification agents, d) separating the plurality of nucleic acid markers and amplification agents from the binding molecule/target molecule complex immobilized on the solid substrate, e) preparing a plurality of amplification mixtures each comprising amplification agents and none or at least one nucleic acid marker molecule, f) subjecting the plurality of amplification mixtures of step e) to conditions that allow amplification of the nucleic acid marker, g) detecting the presence or absence of a signal indicating the amplification of the nucleic acid marker in each of the plurality of amplification mixtures individually and determining the amount of amplification mixtures having a positive signal, and h) comparing the amount determined in step g) with a previously generated standard curve, thereby quantifying the amount of the target molecule in the sample.

In various embodiments, the target molecule is a) directly attached to the solid substrate, or b) attached to the solid substrate by binding to a capture molecule which is attached to the solid substrate. In other various embodiments, the method comprises a washing step between step b) and c). In still other various embodiments, the detection of the amplification of the nucleic acid marker comprises the use of an amplification detection probe.

The non-covalent binding between the binding molecule and the nucleic acid marker of the detection conjugate is formed by streptavidin/biotin or avidin/biotin interaction. In other various embodiments, the amplification agents comprise a nucleic acid primer covalently linked with the release agent.

In still further various embodiments, the amplification is a) a PCR reaction, or b) an isothermal reaction, optionally selected from the group consisting of nucleic acid sequence-based amplification (NASBA), transcription mediated amplification (TMA), Loop-mediated Isothermal Amplification (LAMP), Helicase-Dependent isothermal Amplification (HDA), Recombinase Polymerase Amplification (RPA) and strand displacement amplification (SDA). In other various embodiments, the detection conjugate comprises or consists of a biotinylated antibody, a tetravalent biotin-binding streptavidin (STV) and a biotinylated nucleic acid marker.

In various embodiments the target molecule is an antibody that is attached to the solid substrate by the interaction with an antigen immobilized on the solid substrate and wherein the binding molecule of the detection conjugate is also an antigen of said antibody.

In various embodiments, the preparation of the plurality of amplification mixtures is carried out by using a) droplets/vesicles for encapsulation of the amplification mixture; or b) microcavities.

In further various embodiments, the non-covalent bond between the binding molecule and the nucleic acid is formed in situ at the target molecule.

In a further aspect, a method of releasing a target molecule from a conjugate may occur by using a fusion molecule comprising a nucleic acid primer and a release agent to release the target molecule of the conjugate comprising a nucleic acid molecule and a second molecule, wherein the release agent releases the non-covalent bond by competitive binding to at least one member of the non-covalent bond. In various embodiments, the non-covalent binding between the nucleic acid molecule and the second molecule is formed by streptavidin/biotin or avidin/biotin interaction.

In a still further aspect, a kit may include a) a detection conjugate comprising a binding molecule binding specifically to a given target molecule and a nucleic acid marker, wherein the binding molecule and the nucleic acid marker are linked by non-covalent binding, b) a release agent that releases the non-covalent bond between the binding molecule and the nucleic acid marker of the detection conjugate by competitive binding to at least one member of the non-covalent bond, and c) nucleic acid amplification agents. In various embodiments, the amplification agents of the kit comprise a nucleic acid primer covalently attached to the release agent.

In a fourth aspect, a device to perform the method of the invention may include a) a unit that allows droplet/vesicle preparation, and b) a unit that allows nucleic acid amplification.

### BRIEF DESCRIPTION OF THE DRAWINGS

The invention will be better understood with reference to the detailed description when considered in conjunction with the non-limiting examples and the accompanying drawings.

FIG. 1 shows a schematic diagram of a digital immuno-PCR (IPCR) exemplified by a digital droplet IPCR.

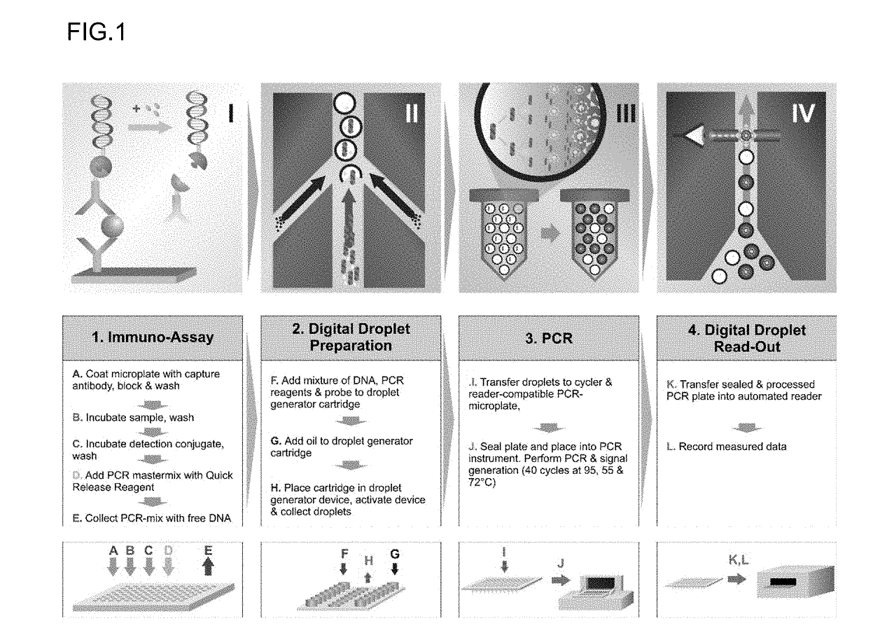

FIG. 2 shows the data of a digital droplet (“DD”) IPCR of interleukin 6 (IL-6).

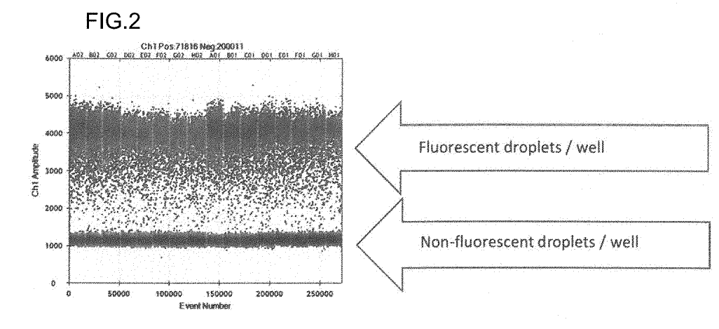

FIG. 3 shows the correlation of a IL-6 dilution series and the amount of DNA marker copies, which was determined by digital droplet analysis (mean of duplicates).

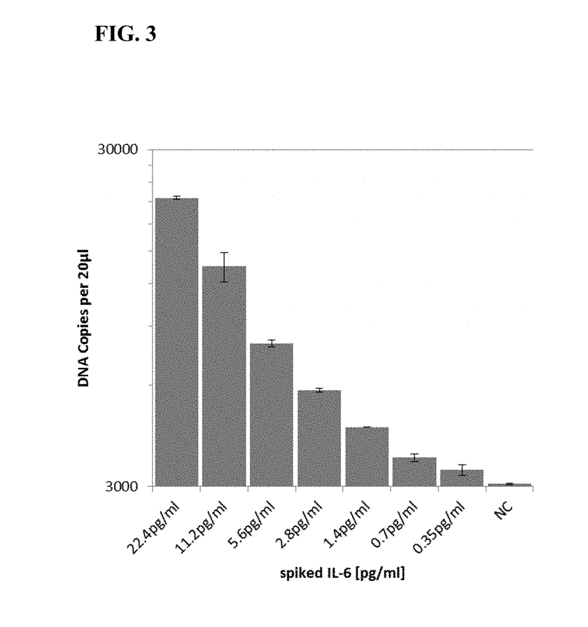

### DETAILED DESCRIPTION

The present inventors surprisingly found that immune-PCR technology and digital-PCR technology can be combined to establish a sensitive and precise method for quantification of target molecules of interest. To combine both technologies, an elution step is required that separates a nucleic acid marker from a detection conjugate/target molecule complex, which is immobilized on a solid substrate. This elution may be achieved by enzymatic or chemical cleavage. However, these reactions require adding of further agents that may interfere with subsequent steps of the method. Alternatively, the nucleic acid marker and the detection conjugate may be cleaved by thermic denaturation. Nonetheless, such reaction is complicated and requires the handling of a hot solution that comprises the risk of contamination (heat generated aerosols may affect contamination).

The present inventors surprisingly found that a nucleic acid marker non-covalent bound to the detection conjugate can be released by a release agent that competitively binds to at least one member of the non-covalent bond. The application of the release agent results in effective and constant elution of the nucleic acid marker, which can be used in subsequent reaction for amplification and quantification. By using a non-covalent bond between the nucleic acid marker and the detection conjugate and by releasing said bond with a competitive binding agent, the above-described disadvantages are overcome.

Therefore, in a first aspect, a method for quantifying a plurality of target molecules in a sample may comprise a) providing (i) the plurality of target molecules, wherein the target molecules are immobilized on a solid substrate, and (ii) a plurality of detection conjugates each comprising a binding molecule binding specifically to the target molecule and a nucleic acid marker, wherein the binding molecule and the nucleic acid marker are linked by non-covalent binding, b) contacting the plurality of target molecules and the plurality of detection conjugates, thereby the detection conjugates bind to the target molecules, c) adding (i) a release agent that releases the plurality of target molecules from the binding molecules and the nucleic acid markers of the plurality of detection conjugates by competitive binding to at least one member of the non-covalent bond, and (ii) nucleic acid amplification agents, d) separating the plurality of nucleic acid markers and amplification agents from the binding molecule/target molecule complex immobilized on the solid substrate, e) preparing a plurality of amplification mixtures each comprising amplification agents and none or at least one nucleic acid marker molecule, f) subjecting the plurality of amplification mixtures of step e) to conditions that allow amplification of the nucleic acid marker, g) detecting the presence or absence of a signal indicating the amplification of the nucleic acid marker in each of the plurality of amplification mixtures individually and determining the amount of amplification mixtures having a positive signal, and h) comparing the amount determined in step g) with a previously generated standard curve, thereby quantifying the amount of the target molecule in the sample.

The terms “quantifying”, and “quantification”, which are used interchangeably herein, refer to processes that relate to or involve the measurement of quantity or the amount of the target molecule or signal of corresponding to the amount of target molecule in a sample (also referred as quantitation), which includes but is not limited to any analysis designed to determine the amounts or proportions of the target molecule or its signal. The quantitatively detection refers to a detection method which provides results describing the amount and type of the target molecule. The quantity can be either an absolute number of copies of the target molecules or a relative amount normalized to a standard amount of the target molecule (for the example, the average amount of the target molecule found in a group of healthy individuals or in a group of specific patients) or normalized to a reference molecule, such as the gene product of a housekeeping gene.

The term “one or more”, as used herein, in connection with molecules relates to at least one, or at least 2, 3, 4, 5, 6, 7, 8, 9, 10, 12, 14, 15, 20, 25 or a plurality of molecules. In this connection, the term “plurality” means more than one, or 2-1000, or 2-100, or even 2-50, or 2-25 or 2-15.

The terms “analyte”, “target compound”, “target molecule” or “target”, as interchangeably used herein, refer to any substance that can be detected with the present method assay by binding to a binding molecule, and which may be present in a sample. Therefore, the analyte can be, without limitation, any substance for which there exists a naturally occurring antibody or for which an antibody can be prepared. The analyte may, for example, be an antigen, a protein, a polypeptide, a hapten, a carbohydrate, a lipid, a metabolite, a cell or any other of a wide variety of biological or non-biological molecules, complexes or combinations thereof. Generally, the analyte will be a protein, peptide, carbohydrate or lipid derived from a biological source such as bacterial, fungal, viral, plant or animal samples. Additionally, however, the target may also be a small organic compound such as a drug, drug-metabolite, dye or other small molecule present in the sample.

The term “sample”, as used herein, refers to an aliquot of material, frequently biological matrices, an aqueous solution or an aqueous suspension derived from biological material. Samples to be assayed for the presence of an analyte by the methods include, for example, cells, tissues, homogenates, lysates, extracts, and purified or partially purified proteins and other biological molecules and mixtures thereof.

Non-limiting examples of samples typically used in the methods include human and animal body fluids such as whole blood, serum, plasma, cerebrospinal fluid, sputum, bronchial washing, bronchial aspirates, urine, semen, lymph fluids and various external secretions of the respiratory, intestinal and genitourinary tracts, tears, saliva, milk, white blood cells, myelomas and the like; biological fluids such as cell culture supernatants; tissue specimens which may or may not be fixed; and cell specimens which may or may not be fixed. The samples used in the methods will vary based on the assay format and the nature of the tissues, cells, extracts or other materials, especially biological materials, to be assayed. Methods for preparing protein extracts from cells or samples are well-known in the art and can be readily adapted in order to obtain a sample that is compatible with the methods.

The term “solid support” or “solid substrate”, as interchangeably used herein, refer to a solid or insoluble substrate/support, commonly a polymeric support, to which a linker moiety (that allows binding of the target molecule or a capture molecule) can be covalently bonded by reaction with a functional group of the support. Many suitable supports are known, and include materials such as polystyrene resins, polystyrene/divinylbenzene copolymers, agarose, and other materials known to the skilled person skilled in the art. It will be understood that an insoluble support can be soluble under certain conditions and insoluble under other conditions; however, for purposes of this invention, a polymeric support is “insoluble” if the support is insoluble or can be made insoluble in a reaction solvent. Further, the solid support may be a soluble or insoluble polymeric structure, such as polystyrene, or an inorganic structure, e.g. of silica or alumina. “Immobilization on the solid substrate” refers to the covalent bond of the target molecule or a capture molecule to said substrate.

The term “detection conjugate” or “detection molecule”, as interchangeably used herein, refers to any molecule or target-binding fragment thereof capable of specifically binding to the target molecule so as to form a specific complex consisting of the detection conjugate and the target. In case of the presence of another binding molecule coupling the detection conjugate/target molecule complex to the solid substrate, the detection conjugate is a second binding molecule used for the specific detection of the analyte. In this case, two binding molecules are used for the specific binding of the analyte in a “sandwich” assay. During sandwich assay, the other binding molecule is termed “capture” molecule. In case of the direct immobilization of the target molecule against a surface without a capture molecule, the detection conjugate is the only binding molecule used for the specific binding of the analyte. In general, the detection conjugate comprises two parts: the first part of the detection molecule allows specific binding of the analyte; the second part comprises or consists of a nucleic acid marker. The “binding molecule”, as used herein, may be an antibody, an antigen (if the target molecule is an antibody), a small molecule, a receptor, a ligand (if the target molecule is a receptor), an aptamer or a lipocalin.

“Specifically binding” and “specific binding”, as used herein, means that the binding molecule binds to the target molecule based on recognition of a binding region or epitope on the target molecule. The binding molecule recognizes and binds to the target molecule with a higher binding affinity than it binds to other compounds in the sample. In various embodiments, “specifically binding” may mean that an antibody or other biological molecule, binds to a target molecule with at least about a 10⁶-fold greater affinity, or at least about a 10⁷-fold greater affinity, or at least about a 10⁸-fold greater affinity, or at least about a 10⁹-fold greater affinity than it binds molecules unrelated to the target molecule. Typically, specific binding refers to affinities in the range of about 10⁶-fold to about 10⁹-fold greater than non-specific binding. In some embodiments, specific binding may be characterized by affinities greater than 10⁹-fold over non-specific binding. In a specific embodiment, the binding molecule uniquely recognizes and binds to the target molecule.

The term “nucleic acid marker” refers to a nucleic acid molecule that will produce a detection product of a predicted size or other selected characteristic when used with appropriately designed oligonucleotide primers in a nucleic acid amplification reaction, such as a PCR reaction. The skilled person is familiar with the design of suitable oligonucleotide primers for PCR and programs are available over the Internet to facilitate this aspect (cf., for example, the http site: bibiserv.techfak.unibielefeld.de/genefisher). A nucleic acid marker may be linear or circular. In non-limiting embodiments, the nucleic acid marker will comprise a predetermined, linear nucleic acid sequence with binding sites for selected primers located at or near each end. In a circular DNA nucleic acid molecule, the primers will be internal rather than at an end, and a single primer may be used, e.g. for Rolling Circle Amplification. Amplified DNA may be detected using any available method, including, but not limited to techniques such as real time PCR, SYBR Green staining, or ethidium bromide staining. In other embodiments, the binding sites for the amplification primers flank an undefined DNA sequence of defined length or which comprises another identifiable characteristic, such as a detectable sequence, in addition to undefined sequences. In some embodiments, the nucleic acid marker are distinguished by the size or mass of the amplified sequences; thus, the DNA sequence between the primers need not be defined as to the exact sequence, just as to the number of bases. Alternatively, the size and/or sequence of the entire nucleic acid marker need not be defined as long as a binding site for a molecular beacon is supplied. In further embodiments, the DNA sequence located between the primer binding sites comprises a “characteristic identification sequence” capable of being detected during the PCR reaction. Fluorescent signal generation may, for example, be sequence-specific (Molecular Beacons, TaqMan, fluorogenic primers, such as the LUX primers (Invitrogen (Carlsbad, Calif.)) or mass dependent (SYBR Green, Ethidium Bromide). In non-limiting embodiments, Molecular Beacons or a TaqMan probe is used to detect the amplification of the nucleic acid marker. Such systems that allow the detection of amplification by generating a specific (fluorogenic) signal are herein referred to as an “amplification detection probe”. The examples provided are not meant to be an exhaustive list of possible nucleic acid detection schemes as those skilled in the art will be aware of alternative markers suitable for use in the methods.

The term “non-covalent”, as used herein, for example in the context of non-covalent binding of the binding molecule to the nucleic acid marker to form the detection conjugate, refers to a bond between two chemical moieties, which is not formed by covalent binding. Examples of different types of non-covalent bonds include, but are not limited to, ion bonds, hydrogen bonds and bonds due to van der Waals forces, Coloumb forces and/or London forces. The non-covalent bond between the binding molecule and the nucleic acid marker may be established by linking each of the above molecules with one member of a binding pair. However, the non-covalent bond may also be formed by indirect non-covalent interaction of functional groups linked to the binding molecule and the nucleic acid marker. One such example is the case, wherein each of the binding molecule and the nucleic acid marker are biotinylated and the non-covalent bond is established by a multivalent streptavidin or avidin molecule. Further non-limiting examples of binding pairs that allow after coupling non-covalent binding between the binding molecule and the nucleic acid marker are two compounds that specifically bind to one another, such as (functionally): a receptor and a ligand (such as a drug), an antibody and an antigen, etc.; or (structurally): protein or peptide and protein or peptide; protein or peptide and nucleic acid; and nucleotide and nucleotide etc. Non-covalent binding pairs include, but are not limited to antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin. Non-covalent binding pairs may also include a c-myc tag, an HA-tag, a T7 tag, a FLAG-tag, a polyhistidine tag (such as (His)₆), a polyarginine tag, a polyphenylalanine tag, a polycysteine tag, or a polyaspartic acid tag and the corresponding interaction partner, such as iminodiacetic acid (Ni-IDA), nitrilotriacetic acid (Ni-NTA), carboxylmethylaspartate (Co-CMA) or an antibody.

The term “contacting”, as used herein, refers generally to providing access of one component, reagent, analyte or sample to another. For example, contacting can involve mixing a solution comprising the detection conjugate with a sample comprising a target molecule. The solution comprising one component, reagent, analyte or sample may also comprise another component or reagent, such as dimethyl sulfoxide (DMSO) or a detergent, which facilitates mixing, interaction, uptake, or other physical or chemical phenomenon advantageous to the contact between components, reagents, analytes and/or samples.

The term “release agent”, as used herein, refers to an agent that allows the separation of the nucleic acid marker from the detection conjugate. The detection conjugate contains a non-covalent bond between the binding molecule and the nucleic acid marker. This bond is separated/weakened by a covalent binding of the release agent to at least one member of the non-covalent bond. In non-limiting embodiments, the release agent comprises or consists of a functional group identical to at least one functional group linked to the binding molecule and/or the nucleic acid marker and forms the non-covalent bond. By providing the release agent in excess over the detection conjugate, the release agent displaces at least one member of the non-covalent bond by competitive binding. In solution, the excess of the release agent over the amount of detection conjugate may be at least 2-fold, 3-fold, 4-fold, 5-fold, 10-fold, 20-fold, 30-fold, 50-fold, 100-fold, 500-fold or 1000-fold. In alternative embodiments, the release agent comprises or consists of a functional group that has an enhanced affinity to at least one member of the non-covalent bond compared to its binding partner linked to the binding molecule and/or the nucleic acid marker. In various embodiments, the functional groups linked to the binding molecule and the nucleic acid marker are not directly binding to each other but their interaction is bridged by a linker molecule. For example, the binding molecule and the nucleic acid marker are each linked to a biotin molecule and their non-covalent binding is formed by a multivalent streptavidin or avidin molecule, which represents the linker molecule. In the above context, the term “competitive binding” refers to the competition between a functional group linked to the binding molecule or the nucleic acid marker and the release agent, called the competitive binding compound, for a limited number of binding sites on a member that forms the non-covalent bond of the detection conjugate. “Members of the non-covalent bond”, as used herein, relates to functional groups that are linked to the binding molecule and the nucleic acid marker to establish their interaction. However, as described above, in various embodiments, the functional groups are not binding directly to each other but each binding to a linker molecule. Also such linker molecule, if present, is considered to be a member of the non-covalent bond. Therefore, in case where the binding molecule and the nucleic acid marker are each linked to a biotin molecule and their non-covalent binding is formed by a multivalent streptavidin or avidin molecule, both biotin molecules and the streptavidin or avidin molecule are regarded as members of the non-covalent bond.

“Nucleic acid amplification” or “DNA amplification”, as interchangeably used herein, refers to any process that increases the number of copies of a specific DNA sequence by enzymatically amplifying the nucleic acid sequence. A variety of processes are known. One of the most commonly used is the polymerase chain reaction (PCR), which is described in U.S. Pat. Nos. 4,683,195 and 4,683,202. PCR involves the use of a thermostable DNA polymerase, known sequences as primers, and heating cycles, which separate the replicating deoxyribonucleic acid (DNA) strands and exponentially amplify a nucleic acid sequence of interest. Any type of PCR, such as quantitative PCR, RT-PCR, hot start PCR, LAPCR₅ multiplex PCR, touchdown PCR, etc., may be used. In various embodiments, real-time PCR is used, in general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence, in PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed. Apart from the template nucleic acid strand, which is the nucleic acid marker, agents that are required to perform a PCR or an isothermal amplification reaction are “nucleic acid amplification agents”. If the nucleic acid marker is amplified by PCR, the nucleic acid amplification agents include, but are not limited to oligonucleotide primer pair, buffer, salts, (DNA) polymerase and deoxynucleoside triphosphate (dNTPs). Such agents are well-known in the art.

The term “separating”, as used herein, refers to physical separation of two elements (e.g., by size or affinity, etc.) as well as degradation of one element, leaving the other intact.

“Preparing a plurality of amplification mixtures”, as used herein, refers to dividing a mixture containing a plurality of eluted nucleic acid markers and nucleic acid amplification agents into a plurality of preparations wherein each preparation comprises nucleic acid amplification agents and none or at least one nucleic acid marker molecule. For the preparation an educated guess or empirical data on the amount of nucleic acid marker can be used to generate amplification mixtures, which contain as the arithmetic mean one nucleic acid marker. Nonetheless, for a plurality of amplification mixtures this means that some mixtures contain none or two or three or even more markers, whereas the majority of mixtures contains one marker. Conditions that allow the amplification of nucleic acids, specifically DNA, independent of the method of amplification (PCR or isothermal amplification reactions) are well-known in the art.

The plurality of target molecules after being detected by immuno-PCR are quantified by so called “digital-PCR”. Typically, one PCR reaction is carried out per sample. However, in digital-PCR (dPCR) the sample is divided into a multitude of sub-samples and the reaction is carried out for each sub-sample individually. This division allows a more reliable and sensitive measurement of nucleic acid marker amounts. For the present method this means that the solution containing the nucleic acid markers is divided into sub-samples as described above, namely by dilution so that each sub-sample/amplification mixture comprises statistically one single nucleic acid marker. As the nucleic acid markers may be distributed not evenly among the plurality of amplification mixtures, each amplification mixture may comprise none, one, two, three, four, five, six, seven, eight, nine, ten or more nucleic acid markers. Each amplification mixture may comprise none or 1-100 nucleic acid markers, or 1-50 nucleic markers, or 1-25 nucleic acid marker, or 1-10 nucleic acid marker, or 1 nucleic acid marker. The dilution may be carried out such that about 50%, or about 60%, or about 70%, or about 80% of the sub-samples contain one single marker nucleic acid molecule, while the remainder contains either none or 2 or more marker molecules.

The term “detection”, as used herein, relates to quantitatively or qualitatively identification of the amplification of the nucleic acid marker (e.g., DNA or RNA) within the amplification mixture. However, in cases in which the amplification is detected quantitatively, the result is recorded as a binary result: this means that for each mixture of the plurality of amplification mixtures a positive or negative detection is recorded. A positive detection refers to a signal that is significantly enhanced over a comparative background signal. A negative signal refers to a signal that cannot be distinguished from a comparative background signal. The absence or presence of the signal based on the amplification of the nucleic acid marker can be detected by several different techniques known in the art. Such techniques may include, but are not limited to fluorescence assay, mass spectrometry, chromatography, Western Blot, or gel electrophoresis. For example, the amplification of the nucleic acid marker may be detected with signals emitted by a TaqMan probe or a Molecular Beacon. In alternative embodiments, the amplification of the nucleic acid marker is determined by gel electrophoresis and ethidium bromide detection.

“Determining the amount of amplification mixtures having a positive signal”, as used herein, may refer to the percentage of amplification mixtures that have a signal that is significantly enhanced over a comparative background signal. The skilled person will understand that the amount of amplification mixtures can also be determined that have a negative signal as this is the reciprocal value of the amount of amplification mixtures having a positive signal.

The term “standard curve”, as used herein, relates to a curve correlating the concentration of a given target molecule in a sample with the amount (e.g. percentage) of positive amplification signals in a plurality of amplification mixtures. Said amplification mixtures are generated from the sample containing the target molecule according to the method steps a) to g).

In various embodiments, the target molecule is a) directly attached to the solid substrate, or b) attached to the solid substrate by binding to a capture molecule which is attached to the solid substrate. In other various embodiments, the method comprises a washing step between step b) and c). In still other various embodiments, the detection of the amplification of the nucleic acid marker comprises the use of an amplification detection probe.

The term “directly attached”, as used herein, refers to target molecules that are bound to a functional group or functional layer of the solid substrate. Alternatively, the target molecule may be attached to a molecule, which is attached to the solid substrate for the mere purpose of binding the target molecule (“sandwich-assay”). Such molecules are herein referred to as “capture molecules” and include, but are not limited to a monoclonal antibody, a group a polyclonal antibodies, an antigen (if the target molecule is an antibody), a small molecule, a receptor (if the target molecule is a ligand) or a ligand (if the target molecule is a receptor).

The term “washing step”, as used herein, refers to a process in which other proteins, lipids, carbohydrates, nucleic acids, and/or other impurities originating from the sample but not the target molecule are removed from the sample. The washing step includes suspension of the target molecule in water, saline and/or lipophilic solution. The solution used for the washing step may be a buffer solution. The washing solution may comprise, for example, a salt, such as NaCl, from about 0.05 to about 0.15 M and/or a lipophilic agent, such as sodium dodecyl sulfate (SLS or SDS), cetyl trimethylammonium bromide (CTAB), octoxinol 9, polysorbate 20, digitonin, dodecyl maltoside, octyl glucoside and surfactants.

The non-covalent binding between the binding molecule and the nucleic acid marker of the detection conjugate may be formed by streptavidin/biotin or avidin/biotin interaction. In other various embodiments, the amplification agents comprise a nucleic acid primer covalently linked with the release agent.

The terms “polynucleotide” and “nucleic acid (molecule)” are used interchangeably to refer to polymeric forms of nucleotides of any length, including naturally occurring and non-naturally occurring nucleic acids. The polynucleotides may contain deoxyribonucleotides, ribonucleotides and/or their analogs. Methods for selection and preparation of nucleic acids are diverse and well-described in standard biomolecular protocols. A typical way would be preparative PCR and chromatographic purification starting from existing template DNAs or stepwise synthesis of artificial nucleic acids.

Nucleotides may have any three-dimensional structure, and may perform any function, known or unknown. The term “nucleic acid molecule” includes single-, double-stranded and triple helical molecules. “Oligonucleotide” refers to polynucleotides of between 3 and about 100, for example 3-50, 5-30, or 5-20 nucleotides of single- or double-stranded nucleic acid, typically DNA.

The term “nucleic acid molecule” or “nucleic acid sequence”, as used herein, relates to DNA (deoxyribonucleic acid) or RNA (ribonucleic acid) molecules. Said molecules may appear independent of their natural genetic context and/or background. The term “nucleic acid molecule/sequence” further refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms.

Oligonucleotides are also known as oligomers or oligos and may be isolated from genes, or chemically synthesized by methods known in the art. A “primer” refers to an oligonucleotide, usually single-stranded, that provides a 3′-hydroxyl end for the initiation of enzyme-mediated nucleic acid synthesis. In non-limiting embodiments, the primer and the release agent, for example the portion containing the functional group binding to a member of the non-covalent bond between the binding molecule and the nucleic acid marker, are linked by a covalent bond, such as σ-bonding, π-bonding, metal to metal bonding, agostic interactions, and three-center two-electron bonds.

The following are non-limiting embodiments of nucleic acids: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, nucleic acid marker and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine. A nucleic acid may also include a backbone modification, wherein the phosphodiester bonds are replaced with phosphorothioates or methylphosphonates.

In still further various embodiments, the amplification is a) a PCR reaction, or b) an isothermal reaction, optionally selected from the group consisting of nucleic acid sequence-based amplification (NASBA), transcription mediated amplification (TMA), Loop-mediated Isothermal Amplification (LAMP), Helicase-Dependent isothermal Amplification (HDA), Recombinase Polymerase Amplification (RPA) and strand displacement amplification (SDA). In other various embodiments, the detection conjugate comprises or consists of a biotinylated antibody, a tetravalent biotin-binding streptavidin (STV) and a biotinylated nucleic acid marker. Other non-limiting examples of detection conjugates are described in U.S. Pat. No. 8,927,210, which is herein incorporated by reference in its entirety.

The term “isothermal amplification”, as used herein, indicates a method of DNA amplification using polymerase chain reaction that uses a single temperature incubation thereby obviating the need for a thermal cycler. By combining with a reverse transcription step, these amplification methods can also be used to isothermally amplify RNA. In several embodiments, the methods and apparatus herein described allow isothermal amplification of a nucleic acid marker. For example, in some embodiments, the isothermal amplification of the marker is performed by the Loop-mediated Isothermal Amplification (LAMP). In some embodiments, the isothermal amplification of a target nucleic acid is performed by Helicase-Dependent isothermal Amplification (HDA). In other embodiments, the isothermal amplification of a target nucleic acid is performed by Recombinase Polymerase Amplification (RPA). In other embodiments, the isothermal amplification is selected from the group consisting of nucleic acid sequence-based amplification (NASBA), transcription mediated amplification (TMA) and strand displacement amplification (SDA).

The target molecule may be an antibody attached to the solid substrate by the interaction with an antigen immobilized on the solid substrate and wherein the binding molecule of the detection conjugate is also an antigen of said antibody.

The term “antibody” is used in the broadest sense and specifically covers monoclonal antibodies as well as antibody variants or fragments (e.g., Fab, F(ab′)2, scFv, Fv diabodies and linear antibodies), so long as they exhibit the desired binding activity (for a review of scFv see Pluckthun (1994) The Pharmacology of Monoclonal Antibodies, Vol. 113. Rosenburg and Moore eds. Springer-Verlag, New York, pp. 269-315). Diabodies are described more fully in, for example, European patent 404097, international patent publication WO 93/11161 and Hollinger et al. (1993) Proc. Natl. Acad. Sci. USA 90: 6444-6448. Linear antibodies are described in Zapata et al. (1995) Protein Eng. 8(10): 1057-1062.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. The monoclonal antibodies can include “chimeric” antibodies (U.S. Pat. No. 4,816,567; and Morrison et al. (1984) Proc. Natl. Acad. Sci. USA, 81: 6851-6855) and humanized antibodies (Jones et al. (1986) Nature, 321: 522-525; Reichmann et al. (1988) Nature, 332: 323-329; Presta (1992) Curr. Op. Struct. Biol. 2: 593-596). A “chimeric” antibody is a molecule in which different portions are derived from different animal species, such as those having a variable region derived from a murine mAb and a human immunoglobulin constant region.

Monoclonal antibodies may be obtained by any technique that provides for the production of antibody molecules by continuous cell lines in culture. These include, but are not limited to the hybridoma technique of Koehler and Milstein (1975), Nature, 256: 495-7; and U.S. Pat. No. 4,376,110), the human B-cell hybridoma technique (Kosbor, et al. (1983), Immunology Today, 4: 72; Cote, et al. (1983), Proc. Natl. Acad. Sci. USA, 80: 2026-30), and the EBV-hybridoma technique (Cole, et al. (1985), in Monoclonal Antibodies And Cancer Therapy, Alan R. Liss, Inc., New York, pp. 77-96). The preparation of monoclonal antibodies specific for a target compound is also described in Harlow and Lane, eds. (1988) Antibodies—A Laboratory Manual. Cold Spring Harbor Laboratory, Chapter 6. Such antibodies may be of any immunoglobulin class including IgG, IgM, IgE, IgA, IgD and any subclass thereof. The hybridoma producing the mAb may be cultivated in vitro or in vivo. Production of high titers of mAbs in vivo makes this a very effective method of production.

“Polyclonal antibodies” are heterogeneous populations of antibody molecules derived from the sera of animals immunized with an antigen, or an antigenic functional derivative thereof. For the production of polyclonal antibodies, host animals such as rabbits, mice and goats, may be immunized by injection with an antigen or hapten-carrier conjugate optionally supplemented with adjuvants.

Alternatively, techniques described for the production of single chain antibodies (U.S. Pat. No. 4,946,778; Bird (1988), Science 242: 423-26; Huston, et al. (1988), Proc. Natl. Acad. Sci. USA, 85: 5879-83; and Ward, et al. (1989), Nature, 334: 544-46) can be adapted to produce gene-single chain antibodies. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)2 fragments that can be produced by pepsin digestion of the antibody molecule and the Fab fragments that can be generated by reducing the disulfide bridges of the F(ab′)2 fragments. Alternatively, Fab expression libraries may be constructed (Huse, et al. (1989), Science, 246: 1275-1281) to allow rapid and easy identification of monoclonal Fab fragments with the desired specificity.

In various embodiments, the preparation of the plurality of amplification mixtures is carried out by using a) droplets/vesicles for encapsulation of the amplification mixture; or b) microcavities.

The formation of droplets/vesicles encapsulating molecules of interest is well-known in the art. Methods describing the formation of droplets are disclosed, for example, by Sharma et al. (Sharma, S. et al. (2012) Microfluidic Diagnostics, volume 949, pages 207-230). Such methods comprise, but are not limited to dielectrophoresis (DEP) and electrowetting on dielectric (EWOD). The term “microcavity”, refers to a solid support that contains a plurality of equal cavities, wherein each of such cavities has a volume of at most 1500 or 1-1000 or 5-500 or 10-100 or 15-50 or 20-30 μl. Such solid support comprising the microcavities may be a thin-film (e.g. a polymer) wherein the cavities are formed by contact-patterning using polydimethylsiloxane stamps (see, for example, Scott, B. M. and Bulović, V. (2012) Photonics Technology Letters, volume 24, pages 104-106). However, the skilled is aware of several alternative methods to produce solid supports comprising microcavities, wherein the solid support may be made of, but not limited to a polymeric, metallic (e.g. gold), glass or hybrid material. The solution containing the nucleic acid marker and the amplification agent can be filled into the microcavities by automated pipetting. The above described droplets and microcavities are used to form a plurality of amplification mixtures. Said plurality of mixtures is prepared from one origin solution. By enlarging or reducing the volume of the amplification mixture each mixture of the plurality only contains one nucleic acid marker (mean value; see also above). The purpose of the droplets or the microvaties is to keep the individual amplification mixtures separate from each other. Therefore, all methods that allow the separation of a plurality of mixtures, such as droplet vaporization, can be used in the method.

In further various embodiments, the non-covalent bond between the binding molecule and the nucleic acid is formed in situ at the target molecule.

The term “in situ”, as used herein, means at the location of the target molecule attached to the solid substrate.

In a further aspect, a method of weakening (or releasing) a non-covalent bond of a conjugate by using a fusion molecule comprising a nucleic acid primer and a release agent to release the nucleic acid molecule from a conjugate comprising a nucleic acid molecule and a second molecule, wherein the release agent may competitively bind to at least one member of the non-covalent bond. In various embodiments, the non-covalent binding between the nucleic acid molecule and the second molecule is formed by streptavidin/biotin or avidin/biotin interaction.

In a still further aspect, the scope encompasses a kit comprising a) a detection conjugate comprising a binding molecule binding specifically to a given target molecule and a nucleic acid marker, wherein the binding molecule and the nucleic acid marker are linked by non-covalent binding, b) a release agent that releases the non-covalent bond between the binding molecule and the nucleic acid marker of the detection conjugate by competitive binding to at least one member of the non-covalent bond, and c) nucleic acid amplification agents. In various embodiments, the amplification agents of the kit comprise a nucleic acid primer that is covalently attached to the release agent.

The term “kit”, as used herein, relates to packaged reagents for quantification of a given target molecule. Accordingly, the kits comprise a detection conjugate, a release agent, and nucleic acid amplification agents. Additionally, such a kit may comprise instructions for use as well as typical reagents known to those skilled in the art.

In a fourth aspect, a device to perform the method of the invention may comprise a) a unit that allows droplet/vesicle preparation, and b) a unit that allows nucleic acid amplification.

A “device”, as used herein, refers to an apparatus that can perform all or some steps of the methods. A unit that allows droplet/vesicle preparation refers to one portion of the device in which droplets can be formed containing the nucleic acid marker and the nucleic acid amplification agent. This unit uses the above describes technologies for the preparation of droplets. A unit that allows nucleic acid amplification refers to another portion of the device which amplifies the nucleic acid marker according to at least one method as described above. In non-limiting embodiments, the two units of the device are interconnected to transfer the droplet containing solution to the amplification unit. Compared to two devices containing the above described units separately, the device provides the advantage of direct and automatic transfer of the droplet solution resulting in more efficiency (less pipetting work) and a minimized risk of contamination. In other non-limiting embodiments, the device comprises a third unit allowing the measurement of the amplification signal. This third unit may be interconnected with the amplification unit to allow the direct and automatic transfer of the amplification mixture.

The term “fusion protein”, as used herein, generally indicates a polypeptide in which heterogeneous polypeptides having different origins are linked, and can refer to a binding molecule or a nucleic acid marker which are linked to functional group, such as biotin, allowing the formation of a non-covalent bond.

The term “sequence”, as used herein, relates to the primary nucleotide sequence of nucleic acid molecules or the primary amino acid sequence of a protein.

The term “conjugate”, as used herein, refers to a compound comprising two or more molecules (e.g., peptides, carbohydrates, small molecules, or nucleic acid molecules) that are chemically linked. The two or molecules desirably are chemically linked using any suitable chemical bond (e.g., non-covalent or covalent bond). Suitable chemical bonds are well-known in the art and include hydrophobic bonds, electrostatic bonds, hydrogen bond, disulfide bonds, acid labile bonds, photolabile bonds, peptidase labile bonds (e.g. peptide bonds), thioether, and esterase labile bonds.

The term “linker” or “linker molecule” refers to a molecule that interconnects two or more functional groups or molecules. The linker molecules according to various embodiments are chemically distinct from the nucleic acid marker and the binding molecule and are capable of binding the binding molecule and the nucleic acid marker and/or other, chemically different linker molecules. To achieve formation of a detection conjugate complex, the linker molecules are at least bivalent, or trivalent, tetravalent, pentavalent, hexavalent or multivalent. In this connection, the term “multivalent” relates to linker molecules that can bind more than 2, or more than 3 other molecules. The multiple molecules bound by the linker molecules may be the same or different. For example, a linker molecule may have binding sites for the nucleic acid marker, the binding molecule and/or another, chemically different linker molecules or, alternatively, 2, 3, 4 or more binding sites for one specific binding partner. In the latter case, complex formation is achieved by coupling one or more binding partner(s) to other components of the conjugate complex, such as the nucleic acid marker, the binding molecule and another, chemically different linker molecules. In this connection, the expression “binding partner” relates to a molecule which is specifically recognized and bound by a linker molecule. The binding partner may thus be a small organic molecule, but can also be any other molecule, such as, for example, a peptide, polypeptide, protein, saccharide, polysaccharide or a lipid or an antigen or hapten. Specific examples for such a pair of linker molecule and binding partner are the streptavidin/biotin and avidin/biotin binding pairs. If the linker molecule is streptavidin/avidin and the binding partner is biotin, the biotin may be coupled to either one or all of the binding molecule, the nucleic acid marker and the second linker molecule to facilitate conjugate complex formation. The binding of the linker molecule to its binding partner and/or the nucleic acid marker, the binding molecule and/or other, chemically distinct linker molecules is non-covalent. The linker molecules may comprise one or more molecules selected from the group consisting of polysaccharides, organic polymers, polypeptides and nucleic acids distinct from the nucleic acid marker. In case the linker molecule comprises a nucleic acid distinct from the nucleic acid marker, the linker molecule may further comprise a polysaccharide, organic polymer or polypeptide chemically coupled to the nucleic acid part.

The present method links the surface-bound IPCR technology and the digital PCR technology requiring a homogeneous liquid phase. The most convenient solution to put the combination of both technologies into practice is the linkage of the IPCR components to small beads, such as microbeads. However, the use of these beads is not compatible with the use of appropriate readout instruments because the micro cannulas and channels of the readout instrument will be clogged with the microparticles.

So a detachment of the nucleic acid marker from the surface is required. For this purpose, three different solutions are possible:

(I) Enzymatic or chemical cleavage of the detection conjugate. The advantage of this method is a precisely controlled reaction (for instance by restriction enzymes and/or proteases). However, the reagents used for cleavage potentially interfere with DNA amplification and thus require an additional and complicated cleaning step.

(II) Thermal denaturation of the detection conjugate. This solution requires no additional reagents. Nonetheless, the handling of hot DNA solution comprises the risk of contamination due to aerosols formed from the hot solution.

(III) Use of a supramolecular detection conjugate that is resolved by the addition of a competing supramolecular binding partner and thereby releases the nucleic acid marker. The advantages of this method are described above in detail. In a nutshell, this method provides a quick, customized, interference-free and mild release of the nucleic acid marker.

### EXAMPLES

### Example: Detection of Interleukin 6 (IL-6) by Means of a Digital Droplet Immuno-PCR Assay (“DD-IPCR”)

Anti-IL-6 capture antibody was immobilized on the surface of a microwell plate (5 μg/ml, 30 μl/well/overnight). The coated surface was then washed, blocked against non-specific interactions, washed again and incubated with a serial dilution series of IL-6 in buffer (22.4-0.35 pg/ml, 30 μl/well, 45 min/RT). For source of reagents and all standard wash and blocking steps, see the manufacturer's instructions of Imperacer Workstation/Imperacer Assay Development KitChimera Biotec; http://www.chimera-biotech.com/technology/literature/(Chimera Biotec GmbH, Dortmund, Germany).

After a further washing step, a detection conjugate comprising previously biotinylated anti-IL-6 antibody, biotin binding tetravalent streptavidin (STV) and biotinylated DNA was added (30 μl/well). The conjugate was incubated for 45 min RT, the wells were washed again and subsequently 30 μl PCR master/well was added. Additionally, the conjugate comprises a DNA-marker specific TaqMan probe and a biotinylated oligonucleotide. After a short incubation (15 min), the liquid was removed from the wells and transferred into a DG8 Cartridge (Bio-Rad, Hercules, Calif., U.S.A.). The cartridge was charged additionally with Droplet Generation Oil (Bio-Rad, Hercules, Calif., U.S.A.) and by using a QX200 Droplet Generator (Bio-Rad, Hercules, Calif., U.S.A.) microvesicles were produced according to the manufacturer's protocol. For a detailed description of the procedure see: http://www.bio-rad.com/de-de/applications-technologies/droplet-digital-pcr-ddpcr-technology.

The droplets were transferred in a PCR compatible microtiter plate, which was sealed, and a standard PCR (according to the protocol of Chimera Biotec GmbH, Dortmund, Germany) was performed in a PCR cycler (Bio-Rad, Hercules, Calif., U.S.A.).

After completion of PCR, the solution of the PCR plate was transferred and measured in a QX200 Droplet Reader (Bio-Rad, Hercules, Calif., U.S.A.). By determining the number of droplets having positive signals vs. the total number of droplets per well, the number of DNA markers before PCR was determined by using the software of the device. The number of DNA markers before PCR was correlated with the interleukin amount used in the IL-6 dilution series to use this as a standard curve to quantify, for example, an unknown amount of IL-6 in a sample.

The invention has been described broadly and generically herein. Each of the narrower species and subgeneric groupings falling within the generic disclosure also form part of the invention. This includes the generic description of the invention with a proviso or negative limitation removing any subject-matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of non-limiting embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

What is claimed is:

1. A method for quantifying a plurality of target molecules in a sample, comprising:
obtaining a solid substrate comprising one or more complexes immobilized on the solid substrate, wherein the one or more complexes comprises:
(i) the plurality of target molecules, and
(ii) a plurality of detection conjugates bound to the plurality of target molecules, wherein each detection conjugate of the plurality of the detection conjugates comprises one or more nucleic acid markers bound to one or more binding molecules,
contacting the one or more complexes immobilized on the solid substrate with one or more release agents to release the one or more nucleic acid markers from the plurality of the detection conjugates; wherein the one or more release agents competitively binds to at least one member of a non-covalent bond of the detection conjugate; and
capturing the one or more nucleic acid markers separated from the one or more complexes immobilized on the solid substrate,
forming one or more solutions with at least the one or more nucleic acid markers and one or more nucleic acid amplification agents,
subjecting the one or more solutions to conditions that allow amplification of the one or more nucleic acid markers, and
detecting whether a signal is present for each solution of the one or more solutions; wherein the signal indicates the presence of the one or more nucleic acid markers in each solution of the one or more solutions where the signal is detected;
determining a number of the one or more solutions having a detected signal; and
comparing the number of the one or more solutions having a detected signal to a pre-determined generated standard curve to quantify the amount of the target molecule in the sample.

2. The method of claim 1, wherein the one or more target molecules are
a) directly attached to the solid substrate, or
b) attached to a capture molecule bound to the solid substrate.

3. The method of claim 1, wherein the method further comprises:
washing the solid substrate prior to contacting the complex immobilized on the solid substrate with the one or more release agents, and/or
detecting the one or more nucleic acid makers in the solution by using an amplification detection probe.

4. The method of claim 1, wherein each detection conjugate comprises the non-covalent bond between the binding molecule and the nucleic acid marker, and wherein the non-covalent bond comprises a streptavidin/biotin interaction, an avidin/biotin interaction, or combinations thereof.

5. The method of claim 1, wherein the one or more amplification agents comprise a nucleic acid primer configured to covalently bond to the one or more release agents.

6. The method of claim 1, wherein the amplification occurs by at least one of:
a) a PCR reaction, and/or
b) an isothermal reaction selected from the group consisting of nucleic acid sequence-based amplification (NASBA), transcription mediated amplification (TMA), Loop-mediated Isothermal Amplification (LAMP), Helicase-Dependent isothermal Amplification (HDA), Recombinase Polymerase Amplification (RPA), strand displacement amplification (SDA), and combinations thereof.

7. The method of claim 1, wherein the plurality of detection conjugates comprises at least one of a biotinylated antibody, a tetravalent biotin-binding streptavidin (STV), a biotinylated nucleic acid marker, and combinations thereof.

8. The method of claim 1, wherein a capture antibody is immobilized on the solid substrate, and wherein the one or more target molecules comprises an antibody interaction with the antigen of the capture antibody.

9. The method of claim 1, further comprising forming one or more solutions into a plurality of amplification mixtures by forming one or more of the following:
a) droplets/vesicles for encapsulation of the amplification mixture;
b) microcavities; and
c) combinations thereof.

10. The method of claim 1, wherein each detection conjugate comprises the non-covalent bond between the one or more binding molecules and the one or more nucleic acid markers; wherein the non-covalent bond is formed in situ at the target molecule.

11. A method comprising:
contacting one or more conjugates with one or more fusion molecules; wherein the one or more conjugates comprises one or more nucleic acid markers bound to one or more second molecules; wherein the one or more fusion molecules comprises one or more nucleic acid primers bound to one or more release agents; and wherein the one or more nucleic acid markers are released from the one or more conjugates by competitive binding to at least one member of a non-covalent bond between the one or more nucleic acid markers and the one or more conjugates; and
capturing at least the one or more nucleic acid markers separated from the one or more conjugates.

12. The method of claim 11, wherein the non-covalent bond between the one or more nucleic acid molecules and the one or more second molecules comprises a streptavidin/biotin and/or avidin/biotin interaction.

13. A kit comprising:
one or more detection conjugates, each detection conjugate comprising a binding molecule and one or more nucleic acid markers; wherein the binding molecule is configured to bind to a target molecule, wherein the binding molecule and the one or more nucleic acid markers are coupled together by a non-covalent bond;
one or more release reagents for releasing at least the one or more nucleic acid markers from the one or more detection conjugates; wherein the releasing occurs by competitive binding of the release agent to at least one member of the non-covalent bond; and
one or more nucleic acid amplification agents.

14. The kit of claim 13, wherein the one or more nucleic acid amplification agents comprises one or more nucleic acid primers covalently bound to the one or more release agents.

15. A device comprising:
a) a unit configured to perform the method of claim 1; and
c) a unit configured to perform nucleic acid amplification.

16. The device of claim 15, further comprising a unit configured to prepare droplets and/or vesicles of the one or more solutions.
